(19) United States
(12) Patent Application Publication
(10) Pub. No.: US 2014/0170725 A1
(43) Pub. Date: Jun. 19, 2014

(54) PROCESS AND APPARATUS FOR CAPTURING GASEOUS AMMONIA

(71) Applicants: DIRK ANDREWS, APTOS, CA (US); RANDOL E. KIRK, ALBUQUERQUE, NM (US); DANIEL F. GORZEN, YPSILANTI, MI (US)

(72) Inventors: DIRK ANDREWS, APTOS, CA (US); RANDOL E. KIRK, ALBUQUERQUE, NM (US); DANIEL F. GORZEN, YPSILANTI, MI (US)

(21) Appl. No.: 14/076,529

(22) Filed: Nov. 11, 2013

Related U.S. Application Data
(63) Continuation-in-part of application No. 13/545,821, filed on Jul. 10, 2012, now abandoned.

(51) Int. Cl. C01C 1/242; C12P 3/00

(52) U.S. Cl. CPC C01C 1/242; C12P 3/00
USPC 435/168; 423/550; 422/162

(57) ABSTRACT

A method and system for collecting gaseous nitrogen compounds into an aqueous solution are provided. The method enables the combination of gaseous sulfur and nitrogen compounds in the aqueous solution to generate ammonium compound components, to include ammonium sulfate. Sulfur may be pressure injected into the solution as gaseous sulfur dioxide. Optionally, carbon may be introduced into the solution as gaseous carbon dioxide. The sulfur may be earlier sourced by a burning of a sulfurous solid. The pH of the solution may be monitored and the introduction of ammonia, carbon and/or sulfur may be halted or constrained while the pH of the solution is measured outside of specified range. The solution may be allowed to age to permit a mix of compounds of ammonium carbonate, ammonium bicarbonate and ammonium carbomate to restabilize and thereby encourage a renewed surge of ammonium sulfate generation.

### CO-PENDING PATENT APPLICATION

This Nonprovisional Patent Application is a Continuation-in-Part Application to Nonprovisional patent application Ser. No. 13/545,821 filed on Jul. 10, 2012 and titled “METHOD FOR PRODUCTION OF ORGANIC AMMONIUM SULFATE USING CAPTURED NH3 AND NH4 PRODUCED BY AEROBIC COMPOSTING”. Nonprovisional patent application Ser. No. 13/545,821 is hereby incorporated by reference in its entirety and for all purposes, to include claiming benefit of the priority date of filing of Nonprovisional patent application Ser. No. 13/545,821.

### FIELD OF THE INVENTION

The present invention generally relates to the remediation of areas and materials that present undesirable levels of nitrogen compounds, ammonia and/or ammonium compounds. More particularly, the present invention is directed to capturing gaseous ammonia in an aqueous solution by precipitation and/or conversion into non-volatile ammonium compounds and/or ammoniacal salts.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Various natural processes, agricultural activities and sewage treatment operations generate outputs that include nitrogen compounds, ammonium compounds and ammonia, wherein the ammonia is generated in a gaseous state and/or out-gasses at ambient temperatures. Factory farming of livestock, offered as one example of a relevant industrialized agricultural activity that often generates excessive levels of nitrogen compounds and ammonia gas, is increasingly drawing attention as a source of nitrogen compounds pollution of soil, water and air. It is noted that both mammalian dung and avian feces contain nitrogen compounds that can contribute to pollution of the natural environment. Sewage treatment plants are also generally tasked with reducing or eliminating human contribution to nitrogen compounds pollution of the environment. In an additional area relevant to certain applications of the present invention, sites of drug and chemical manufacture can be contaminated by ammonia gas and other compounds containing nitrogen.

The prior art provides methods of capturing ammonia by generating concentrated sulfuric acid solutions and transporting the concentrated acidic solution to a site where a target gaseous ammonia is located. The concentrated sulfuric acidic solution sulfuric is then mixed with a water volume to create an acid bath that is exposed to the target ammonia. This prior art method includes several short comings, not the least of which are the costs of handling and transportation and the risk of metal contamination of the concentrated sulfuric acid solution during storage and transit.

Yet the prior art fails to provide optimal methods and systems that enable the extraction of nitrogen from gaseous ammonia and ammonium compounds present in outputs of many widely practiced industrial and agricultural systems.

There is therefore a long-felt need to provide a method and apparatus that enable the collection of nitrogen compounds from laboratory facilities, industrial sites and agricultural operations.

### OBJECTS OF THE INVENTION

It is an object of the method of the present invention (hereinafter, “the invented method”) to remove gaseous ammonia from a site atmosphere by introducing sulfur dioxide as a solute to acidify an aqueous solution, wherein the sulfur dioxide is generated by burning sulfur on-site, and whereby the resultant acidic aqueous solution absorbs the gaseous ammonia and generates resultant chemical compounds that capture nitrogen from nitrogen compounds.

It is an optional object of the invented to generate ammonium sulfate as a resultant compound of interaction sponsored within the acidic aqueous solution as a result of absorption of ammonia by the aqueous solution, and optionally absorbing carbon dioxide, from the site atmosphere.

It is an additional optional object of the invented method to absorb ammonia and optionally absorbing carbon dioxide in the acidic aqueous solution, wherein the ammonia and the carbon dioxide is generated by bacterial processing of organic waste matter.

It is a still additional optional object of the invented to generate ammonium sulfate as a resultant chemical compound of interaction of the acidic aqueous solution with ammonia, wherein the ammonia is generated by bacterial processing of organic waste matter.

### SUMMARY

Toward these and other objects that are made obvious in light of the present disclosure, a the invented method and an invented apparatus are provided that enable the extraction of nitrogen from gaseous ammonia by the application of sulfur dioxide generated by burning sulfur. In an optional aspect of the invented method, gaseous ammonia is introduced into an acidic aqueous solution and ammonium sulfate is produced from the resulting aqueous solution. Sulfur and/or sulfur dioxide may be introduced into the aqueous solution to further acidify the aqueous solution and sponsor the production of ammonium sulfate. Optionally of additionally, carbon and/or carbon dioxide may be introduced into the aqueous solution to further sponsor the production of ammonium sulfate.

In a first application of the invented method, a volume of source air that comprises gaseous ammonia is introduced into an aqueous solution containing sulfur dioxide. The source air containing the ammonia gas may optionally simply be introduced into the water volume without filtering out of any constituents and/or without any significant or intended chemical processing.

In another optional aspect of the invented method, the internal atmosphere of an enclosed structure containing ammonia gas and optionally carbon dioxide is at least partially scrubbed of the ammonia gas by exposing the enclosed internal atmosphere to an acidic aqueous solution. The aqueous solution preferably comprises sulfur dioxide generated by burning sulfur in the presence of oxygen. The acidified aqueous solution having received the sulfur dioxide then is exposed to gaseous ammonia to sponsor the production of chemical compounds within the aqueous solution whereby gaseous ammonia and nitrogen compounds are removed from the internal atmosphere. Ammonium sulfate may be produced as a resultant compound in certain alternate preferred embodiments of the invented method.

In another optional aspect of the invented method, an enclosure is established at a site contaminated with a solid or liquid source material, wherein the source material contains ammonium compounds and emits gaseous ammonia. The enclosure may be a portable structure that is temporarily erected as the instant site and may be successively redeployed at alternate locations. Emission of ammonia gas may be facilitated or accelerated by aerating the source material, e.g., mechanically tilling solid source material, or churning a liquid source material with ambient air containing oxygen. A resultant acceleration of gaseous ammonia production by disturbance and/or introduction of oxygen into the source material may be effected by the organic function of bacteria present or seeded within the source material.

In yet another optional aspect of the invented method, the pH of the aqueous solution may be monitored and the introduction of ammonia, sulfur dioxide and/or carbon dioxide may be halted while the pH is measured outside of a prespecified range, e.g., a range of preferably from approximately 4.0 to 5.0, or alternately a range of from 3.0 to 6.0.

In a still additional optional aspect of the invented method, ammonium sulfate is filtered out and/or extracted from the aqueous solution and optionally provided for or used as an agricultural fertilizer. The ammonium sulfate may be removed from the aqueous solution as a concentrated solution or in combination with a portion of the aqueous solution.

In an even other optional aspect of the invented method, gaseous sulfur dioxide is pressure injected and/or infused into the aqueous solution to sponsor formation of solid ammonium sulfate, other precipitates, and/or chemical components.

In another optional aspect of the invented method, components are removed from the aqueous solution and the resultant water is reused in a following cycle of scrubbing gaseous ammonia from an enclosed atmosphere and/or ammonium sulfate generation.

In a still other optional aspect of the invented method, the aqueous solution may be allowed to age to permit a mix of compounds within the aqueous solution, including but not limited to ammonium carbonate, ammonium bicarbonate and ammonium carbomate, to rebalance and thereby sponsor a renewed surge of ammonium sulfate generation.

This Summary and Objects of the Invention is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

These, and further features of the invention, may be better understood with reference to the accompanying specification and drawings depicting the preferred embodiment, in which:

FIG. 1A is a process chart comprising aspects of the invented method;

FIG. 1B is a block diagram of a first preferred embodiment of the invented apparatus (hereinafter, “first system”) coupled with a gaseous ammonia source, the first system comprising a reaction chamber holding a water volume, an ammonia gas scrubber module coupled with the reaction chamber and the gaseous ammonia source, a sulfur dioxide module coupled with the reaction chamber and providing sulfur dioxide to the water volume, and a combined reverse osmosis module and electrodialysis coupled with the reaction chamber;

FIG. 1C is a block diagram of a second preferred embodiment of the invented apparatus (hereinafter, “second system”) coupled with a gaseous ammonia source, the second system comprising the first system and an agitator module coupled with a source material, the agitator module adapted to encourage production of gaseous ammonia by the gaseous ammonia source;

FIG. 1D is a block diagram a first preferred embodiment of the of the ammonia gas scrubber module of FIG. 1B;

FIG. 1E is a block diagram of a first preferred embodiment of the sulfur dioxide module of FIG. 1B;

FIG. 1F is a block diagram a first preferred embodiment of the combined reverse osmosis module and electrodialysis of FIG. 1B;

FIG. 2 is a flow chart of a first preferred embodiment of the invented method (hereinafter, “first method”) that may be implemented by the first system of FIG. 1B and having optional aspects that may be implemented by the second system of FIG. 1C;

FIG. 3 is a cut-away top view of the first system adapted to decontaminate an internal volume of air of a substantively enclosed and contaminated structure, wherein the enclosed air includes gaseous ammonia;

FIG. 4 is a cut-away side view of the first system adapted to remediate a liquid spill, wherein a portable tent source enclosure is placed above and around the liquid spill and an air pump is placed and positioned to pump air into the liquid source material in order to sponsor an accelerated production of source ammonia from the liquid spill by bacterial action;

FIG. 5 is an example of the first system enclosing an accumulation of a substantively solid source material that emits ammonia gas, wherein the first system is augmented with a rotatiller applied to agitate the solid source material and accelerate the production and capturing of gaseous ammonia;

FIG. 6 is a schematic diagram of an optional internal control system of the first system of FIG. 1A with optional modules that extend control to the second system of FIG. 1B;

FIG. 7 is a schematic diagram of a controlled power distribution network of the control system of the first system of FIG. 1A and including optional elements of the second system of FIG. 1B;

FIG. 8 is a cut-away view of elements of the first system of FIG. 1 that direct gaseous ammonia from the enclosure of the source material and to delivery within the reaction chamber;

FIG. 9 is a cut-away view of the distribution system for sulfur dioxide within the reaction chamber of the first system of FIG. 1;

FIG. 10 is a block diagram of a third alternate preferred embodiment of the present invention (hereinafter, “third system”) wherein a source of pressurized carbon dioxide is provided and is adapted to deliver gaseous carbon dioxide into the water volume and within the reaction chamber of the first system of FIG. 1;

FIG. 11 is a cut-away view of the distribution system for carbon dioxide within the reaction chamber of the third system of FIG. 11;

FIG. 12 is an illustration of a motorized embodiment of the first system of FIG. 1B; and

FIG. 13 is an illustration of a carbon dioxide generation module that accepts mammalian exhalation a source of gaseous carbon dioxide as coupled with the third system of FIG. 10.

### DETAILED DESCRIPTION OF THE INVENTION

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

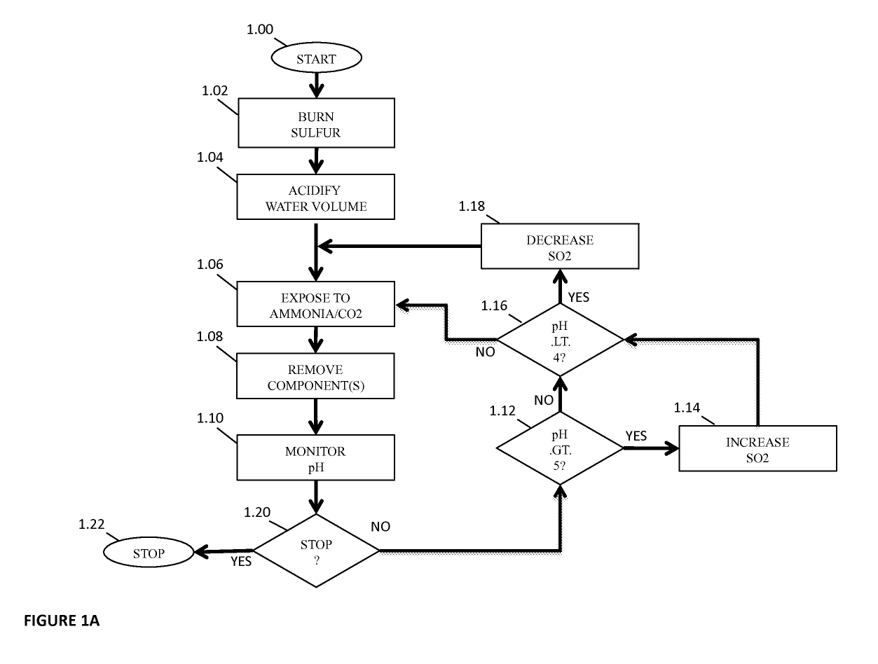

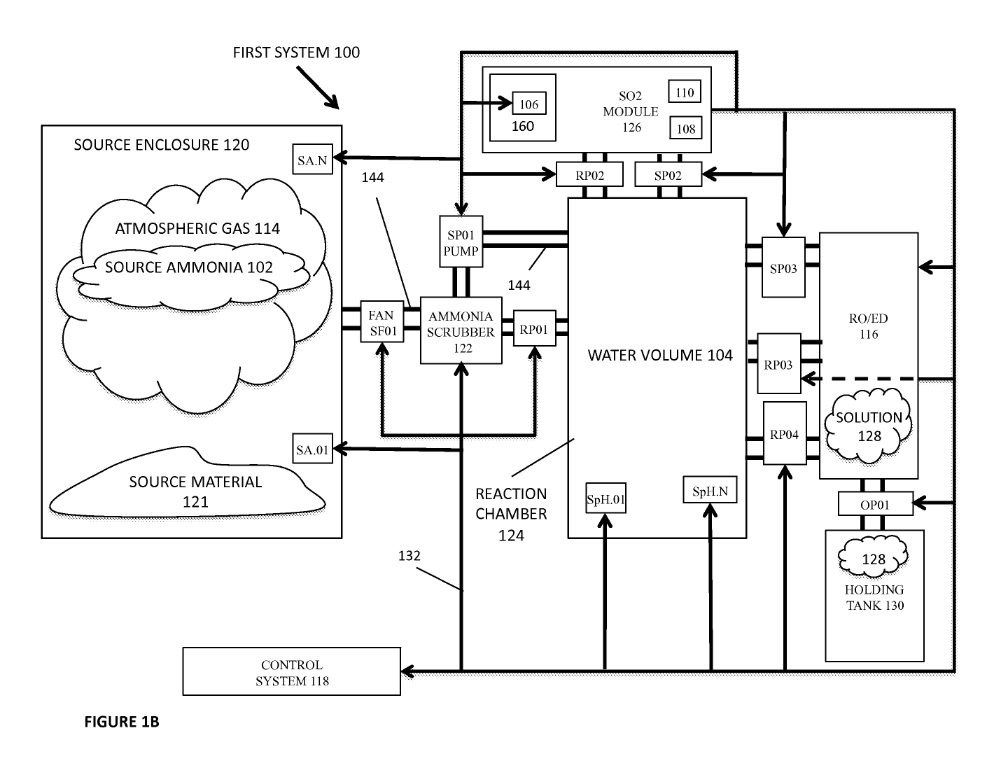

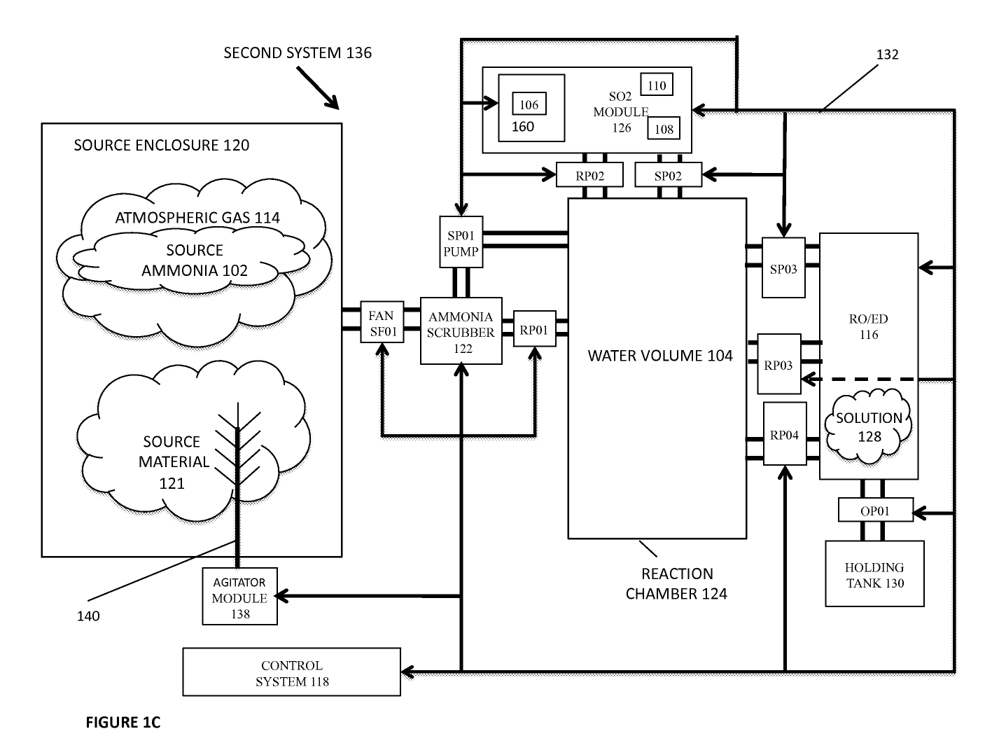

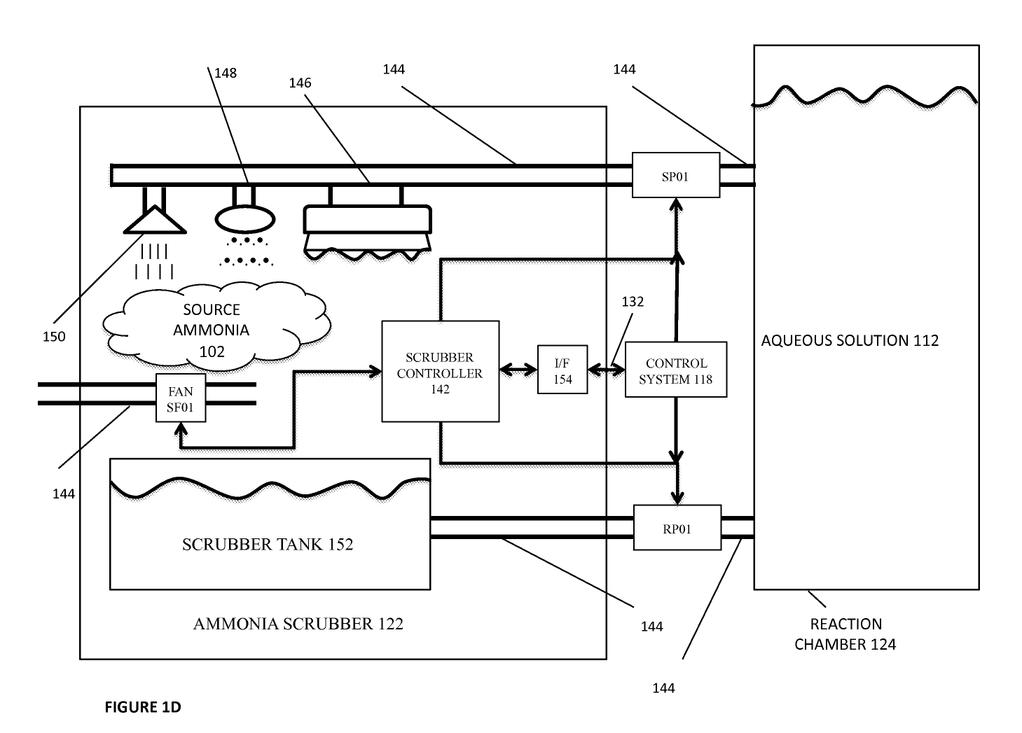

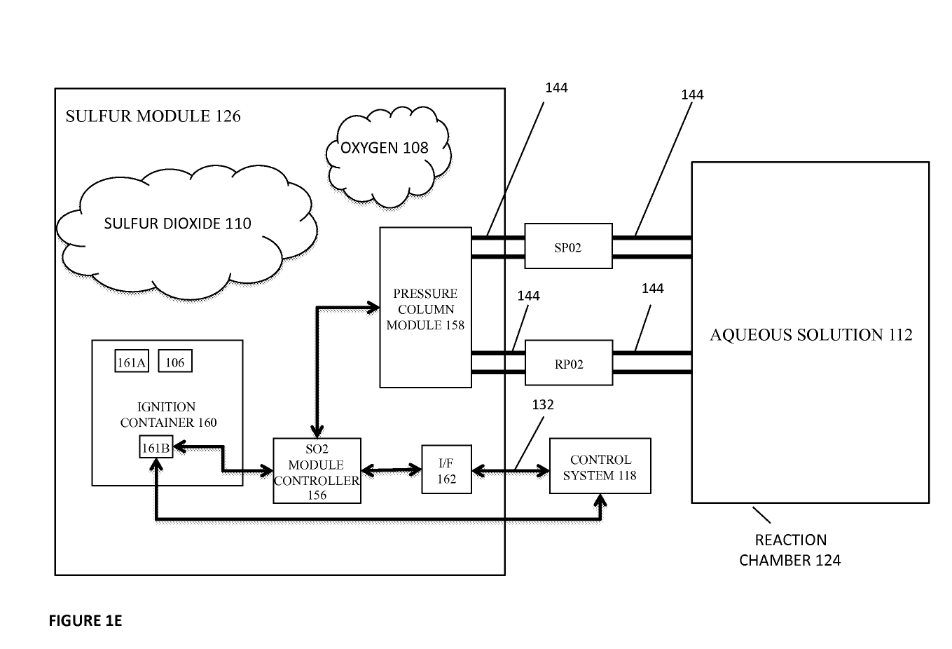

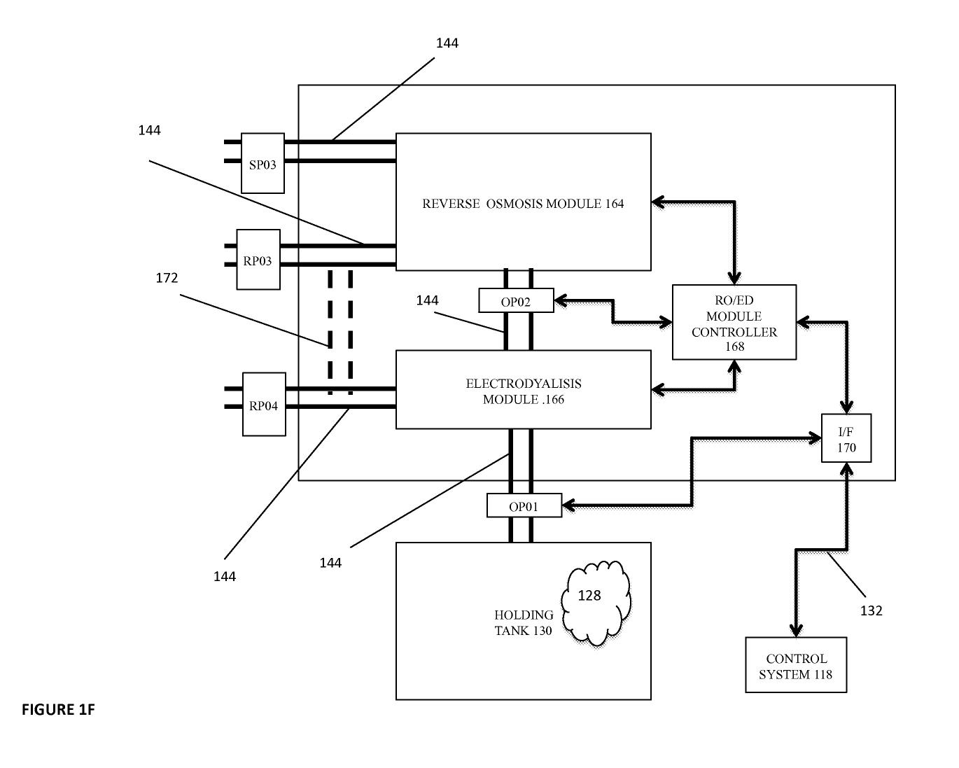

Referring now generally to the Figures and particularly to FIGS. 1A, 1B and 1C, FIG. 1A is a process chart comprising aspects of the invented method that may be instantiated by the first preferred embodiment 100 of the present invention (hereinafter, “the first system 100”). In step 1.00 the first system 100 is preferably co-located with a volume of source ammonia gas 102. In step 1.02 and proximate to a water volume 104, a sulfur mass 106 in a solid form is burned in the presence of atmospheric oxygen 108 to form gaseous sulfur dioxide 110. The temperature of the source ammonia gas 102 and the water volume 104 is initially preferably within 5 degrees Celsius of the ambient temperature of the site environment of the first system 100 and also preferably within the temperatures range of greater than the freezing point of the water volume 104 and less than the boiling point of the water volume 104.

The water volume 104 is then acidified in step 1.04 by introduction of the sulfur dioxide 110 to form an acidic aqueous solution 112, as indicated in FIG. 1C.

The acidic aqueous solution 112 is then exposed to the source ammonia gas 102, and optionally carbon dioxide, in step 1.06, wherein portions of the source ammonia gas 102, and optionally gaseous carbon dioxide, is absorbed by the aqueous solution 112 in step 1.06. It is understood that the source ammonia gas may 102 may be comprised within an enclosed atmospheric gas 114 that includes other atomic and molecular components, such as carbon dioxide, and that the acidic aqueous solution 112 may absorb carbon dioxide and additional molecules and free atoms from the enclosed atmospheric gas 114 in step 1.06. It is further understood that the enclosed atmospheric gas 114 may be formed by adding ammonia, carbon dioxide and other products of bacteria acting on organic waste, e.g., dung or feces, to a pre-existing ambient atmosphere.

Precipitates, other solutes and/or certain non-aqueous components of the aqueous solution 112, e.g., ammonium sulfate, are concentrated and collected by circulation through a collection module 116 in step 1.08 to form an output solution 128 that is held in a holding tank 130 for removal from the first system 100, and the pH of the aqueous solution 112 is monitored in step 1.10. When a pH measurement of greater than 5.0 is determined in step 1.12, the rate of volumetric exposure of the gaseous sulfur dioxide 110 is increased in step 1.14, and when a pH measurement of lower than 4.0 is determined in step 1.16, the rate of volumetric exposure of the gaseous sulfur dioxide 111 is decreased in step 1.80

It is understood that the aqueous solution 112 is preferably substantively and continuously exposed to gaseous sulfur dioxide 110 in steps 1.06 through 1.18 albeit possibly at varying rates of volumetric exposure to the sulfur dioxide gas 110 is increased in step 1.14. It is further understood that the absorption of the source ammonia gas 102 indicated in step 1.06 and the collection of solutes and non-aqueous components of the aqueous solution of step 1.08 are preferably continuously and contemporaneously occurring during the instantiation of the loop of steps 1.06-1.18.

An operator or an automated control system 118 may act and/or elect to stop the process loop of steps 1.02 through 1.18 in step 1.20 whereby the burning of the sulfur mass 106 and the processes of steps 1.04 through 1.18 are halted are minimized.

Referring now generally to the Figures and particularly to FIG. 1B, FIG. 1B is a block diagram of the first system 100 that includes certain optional elements. The first system 100 is coupled to a source enclosure 120 that contains the source ammonia gas 102 and the enclosed atmospheric gas 114. The source ammonia gas 102 may be emitted from a source material 121 containing ammonium and/or ammonium compounds, and the first system 100 is adapted to withdraw the source ammonia gas 102, by itself and/or mixed within the enclosed atmospheric gas 114 located within the source enclosure 120, into an ammonia scrubber module 122 through which the water volume 104 is circulated. The water volume 104 is maintained within a reaction chamber 124 and the ammonia scrubber module 122 (hereinafter, “the ammonia scrubber module 122”) is coupled to both the source enclosure 120 and the reaction chamber 124 and is further adapted to circulate the aqueous solution 112 to absorb the source ammonia gas 102. The source ammonia gas 102 is thus removed from the source enclosure 120 and inserted into the water volume 104 as the water volume 104 is circulated through the ammonia scrubber 122. A sulfur dioxide generation module 126 is also coupled with the reaction chamber 124 and is adapted to insert or infuse the gaseous sulfur dioxide 110 into the water volume 104 to form the aqueous solution 112 as the water volume 104 is circulated through the sulfur dioxide generation module 126. The collection module 116 is a combined reverse osmosis module and electrodialysis module 116 (hereinafter, “the RO/ED module 116”) and is additionally coupled with the reaction chamber 124 by tuning 144 to withdraw aqueous solution 112 and preferably return water volume 104. The RO/ED module 116 is adapted to remove certain chemicals, e.g., ammonium sulfate, from the aqueous solution 112 as the aqueous solution 112 is circulated through the RO/ED module 116. A concentrate output holding tank 126 is coupled with the RO/ED module 116 and is adapted to receive a concentrated output solution 128 formed within the RO/ED module 116 and containing both (a.) a portion of the water volume 104 as a solvent and (b.) a solute or component of at least one type of resultant chemical, e.g., ammonium sulfate, formed within the aqueous solution 112 by the invented method. The aqueous solution 112 and the concentrated output solution 128 may thus include ammonium sulfate as a solute or component, whereby ammonium sulfate is produced in a manner that is in conformance one or more governmental, regulatory or organizational standards and the resultant ammonium sulfate may receive a certification of a preferred or particular origin, such as a being certified, graded, trademarked or marked as a special type of organic sulfate. It is understood that the receipt of such certifications or authorizations may increase the market value and perceived quality of the resultant ammonium sulfate of the concentrated output solution 128.

It is also understood that the first system 100 may include commercially available equipment or their equivalents, wherein the ammonia scrubber 122 may be or comprise, or be comprised within, a wet flue gas scrubber marketed by Deryck A Gibson Ltd. of Kingston Jamaica. In various alternate preferred embodiments of the present invention, the acidified aqueous solution 112 may presented to the source ammonia gas 102 within the ammonia scrubber 122 as a mist, a spray or a waterfall as the aqueous solution 112 is circulated through the ammonia scrubber 122. The reaction chamber 124 may comprise sheets, walls, a bottom wall and or/ceiling wall of polyvinyl chloride or other suitable material known in the art.

The sulfur dioxide module 126 may be or comprise, or be comprised within, a sulfur dioxide burner system as marketed by Harmon Systems International, LLC of Bakersfield, Calif., whereby the sulfur dioxide gas 110 may be generated and commingled with water volume 104 that is circulated through the sulfur dioxide module 126. It is understood that the Harmon sulfur dioxide burner system oxidizes sulfur 106 into sulfur dioxide gas 110 by burning the elemental sulfur 106 with a propane torch in the presence of a pressurized circulating portion of the water volume 104 and air containing oxygen 108. The sulfur dioxide gas 110 is combined with the water volume 104 to produce sulfurous acid, or H2SO3, within the aqueous solution 112

In addition, the RO/ED module 116 may be or comprise, or be comprised within, a reverse osmosis/electrodialysis system as marketed by Ameridia Corporation of Moerdjik, Netherlands.

A control module 200 of the first system 100 generates and communicates commands to direct the activity, and provides electrical power that enables the functioning, of the first system 100 in the removal gaseous ammonia and the generation of resultant chemical compounds and precipitates e.g., ammonium sulfate. A communications and power bus 132 of the control module 118 enables the control module 118 to send and receive commands and data within the first system 100 and selectively and controllably provide power to other modules 116 122, 126, supply fans SF01, supply pumps SP01-SP04, motorized fluid return pumps R01-R03, and an output pump OP01.

Referring now generally to the Figures and particularly to FIG. 1C, FIG. 1C is a block diagram of a second preferred embodiment of the invented system 136 (hereinafter, “the second system 136”) that includes the first system 100 and an agitator module 138 having an effector 140. The effector 140 is positioned relative to the source material 121 and is adapted to agitate the source material 121 in order to sponsor bacterial activity that accelerates a production of the source ammonia gas 102 for capture within the enclosure. The agitator module 140 may be or comprise (a.) a motorized rotatiller, wherein the effector 140 is or comprises a mechanical arm or rake that is motor driven to mechanically disturb and aerate the source material 121; (b.) a pressurized air pump, wherein the effector 140 is or comprises a gas hose that delivers pressurized ambient air into the source material 121 and thereby disturbs and aerates the source material 121 with the pressurized ambient air.

Referring now generally to the Figures and particularly to FIG. 1D, FIG. 1D is a detailed block diagram of a first preferred embodiment of the ammonia scrubber 122. A first fluid supply pump SP01 as energized by a scrubber system control module 142 and/or the control system 118 pumps portions of the aqueous solution 112 from the reaction chamber 124 through substantively chemically inert tubing 144 through one or more aeration fixtures 146-150 to enable the aqueous solution 112 to absorb the source ammonia 102. The ammonia scrubber 122 further comprises a scrubber interface 154 that is bidirectionally communicatively coupled with the control module 118 via the communications and power bus 132. The scrubber interface 154 is additionally bi-directionally communicatively coupled with, or comprised within, the scrubber system control module 142.

The aqueous solution 112 passes through the source ammonia gas 112 and falls by gravity into a scrubber tank 152. A first aeration fixture 146 releases the aqueous solution 112 within the ammonia scrubber 122 as a sheet of fluid. A second aeration fixture 148 is a showerhead that releases the aqueous solution 112 into the source ammonia gas 102 as a fine water mist. A third aeration fixture 150 is a showerhead that releases the aqueous solution 112 into the source ammonia gas 102 as water droplets.

A first motorized fluid return pump SP01 as energized by the scrubber system control module 142 and/or the control system 118 pumps the aqueous solution 112 captured by the scrubber tank 152 through additional tubing 144 and thereby returns the aqueous solution 112 to the reaction chamber 124. An optional first supply fan SF01 as energized by the scrubber system control module 142 and/or the control system 118 propels or drives the source ammonia gas 102 and the enclosed atmospheric gas 114 from the enclosure 120 and into the ammonia scrubber 122 through a length of tubing 144. The scrubber tank 152 may comprise sheets, walls, a bottom wall and/or ceiling wall of polyvinyl chloride or other suitable material known in the art.

It is understood that the ammonia scrubber 122 may be or comprise a suitable and commercially available gas scrubber known in the art, and that the source fan SF01, the first motorized fluid supply pump SP01 and/or the first motorized fluid return pump RP01 may be comprised within the ammonia scrubber 122. It is further understood that the tubing 144 may be or comprise polyvinyl chloride piping or other suitable and preferably substantively chemically inert material known in the art.

Referring now generally to the Figures and particularly to FIG. 1E, FIG. 1E is a detailed block diagram of a first preferred embodiment of the sulfur dioxide module 126. A second motorized fluid supply pump SP02 as energized by an SO2 module control module 156, and/or the control system 118, and thereupon pumps and circulates portions of the aqueous solution 112 from the reaction chamber 124 through substantively chemically inert tubing 144 through a pressure column module 158. The pressure column module 158 creates a pressure differential that infuses and/or introduces sulfur dioxide gas 110 into the water volume 104 to form and acidify the aqueous solution 112. An ignition chamber 160 is adapted to maintain the sulfur 106 within the sulfur module 126 before and during of the ignition of the sulfur 106. The ignition of the sulfur 106 may be accomplished by a user manually applying a flame 161 to the sulfur 106 or by an electronically controlled ignition device 161B that (a.) issues a flame or an igniting spark to the sulfur 106 when energized, or (b.) receives an ignition command message from the SO2 module controller 156 and/or the control system 118 and is thereby directed to generate a spark, a blue flame and/or another sulfur ignition medium known in the art. Still alternatively, optionally or additionally, the sulfur 106 may be or comprise touch-to-burn sulfur and may be manually ignited.

An SO2 module interface 162 is disposed between, and bi-directionally communicatively coupled with both of, the control system 118 and the SO2 module controller 156. Bi-directional communications between the control module 200 and the SO2 module controller 156 are enabled by the communications and power bus 132 and the SO2 module interface 162, whereby commands and data may be communicated to and from the control module 200 and the SO2 module controller 156. Electrical power is also provided to the sulfur dioxide module 126 via the communications and power bus 132 and the SO2 module interface 162.

Optionally and alternatively electrical power and/or commands are provided electronically controlled ignition device 161B by a communicative coupling of the electronically controlled ignition device 161B with the SO2 module controller 156 and/or the power and communications bus 132 of the control system 118.

It is understood that the sulfur module 126 may be or comprise a sulfur burner as marketed by Harmon Systems International, LLC of Bakersfield, Calif., or other suitable sulfur burner known in the art. It is further understood that the sulfur burner 126 may be or comprise a suitable and commercially available sulfur burner known in the art, and that the second motorized fluid supply pump SP02 and/or the second motorized fluid return pump RP02 may be comprised within the sulfur burner 126.

FIG. 1F is a block diagram a first preferred embodiment of the RO/ED module 116. The RO/ED module 116 may include a reverse osmosis module 164, an electro-dialysis module 166, a reverse osmosis electrodialysis electronic logic controller module 168 (hereinafter, “RO/ED controller 168”), an electronic interface 170 to the reverse osmosis electrodialysis electronic logic controller module (hereinafter, “RO/ED interface 170”) and a second motorized fluid output pump OP02. It is understood that one or more of the third motorized fluid supply pump SP03, the third motorized fluid return pump RP03, the fourth motorized fluid return pump RP04, the first motorized fluid output pump OP01, and the output holding tank 130 may be optionally or additionally comprised within the RO/ED module 116. Bi-directional communications between the control module 200 and the RO/ED controller 168 is enabled by the communications and power bus 132 and the RO/ED interface 170, whereby commands and data may be communicated to and from the control module 200 and to the RO/ED controller 168. Electrical power is also provided to the RO/ED module 116 via the communications and power bus 132 and the RO/ED interface 170. The RO/ED controller 168 is optionally bidirectionally communicatively coupled to the reverse osmosis module 164 and may provide required electrical power and control signals to the reverse osmosis module 164 that direct and enable the reverse osmosis module 164 to substantively extract water volume from the aqueous solution 122 by reverse osmosis. The RO/ED controller 168 is further optionally bidirectionally communicatively coupled to the electro-dialysis module 166 and may provide required electrical power and control signals to the electro-dialysis module 166 that direct and enable the electro-dialysis module 166 to substantively extract additional water volume from the aqueous solution 122 by electrodialysis. The aqueous solution 112 is delivered to the reverse osmosis module 164 by energizing the third motorized fluid supply pump SP03 via a length of tubing 144. After some water volume 104 is extracted from the aqueous solution 112 by the reverse osmosis module 164, the resultant aqueous solution 112 is delivered to the electrodialysis module 166 from the reverse osmosis module 164 by energizing the second motorized fluid output pump OP02. The RO/ED controller 168 is additionally optionally electrically coupled to the second motorized fluid output fluid pump OP02 and selectively provides electrical power to energize the second motorized fluid output fluid pump OP02 to enable transfer of the aqueous solution 112 from the reverse osmosis module 164 and to the electrodialysis module 166.

The RO/ED controller 168 and/or the control module 200 may optionally or additionally be coupled to the third motorized fluid supply pump SP03 and/or the third motorized fluid return pump RP03 and selectively energize the third motorized fluid supply pump SP03 and/or the third motorized fluid return pump RP03 to enable a delivery of the aqueous solution 112 to the reverse osmosis module 164 and return of water volume 104 from the reverse osmosis module 164 to the reaction chamber 124. The RO/ED controller 168 and/or the control system 118 may further optionally or additionally be coupled to the fourth motorized fluid return pump RP04 and selectively energize the fourth motorized fluid return pump RP04 to enable a return of water volume 104 from the electrodialysis module 166 to the reaction chamber 124. The RO/ED controller 168 and/or the control system 118 may further optionally or additionally be coupled to the first motorized fluid output pump OP01 and selectively energize the first motorized fluid output pump OP01 to enable transfer of the output solution 128 from the electrodialysis module 166 to the holding tank 130. The holding tank 130 may be or comprise one or more walls, floor wall, and/or ceiling comprising polyvinyl chloride or other suitable material known in the art.

An optional or additional RO/ED tubing length 172 may couple the reverse osmosis module 164 and the fourth motorized fluid return fluid pump RP04 and may enable the fourth motorized fluid return fluid pump RP04 to drive water volume from both the reverse osmosis module 164 and the electrodialysis module 166 and into the reaction chamber 124. The RO/ED tubing length 172 may be or comprise perforated polyvinyl chloride piping and/or other suitable and substantively chemically inert material known in the art.

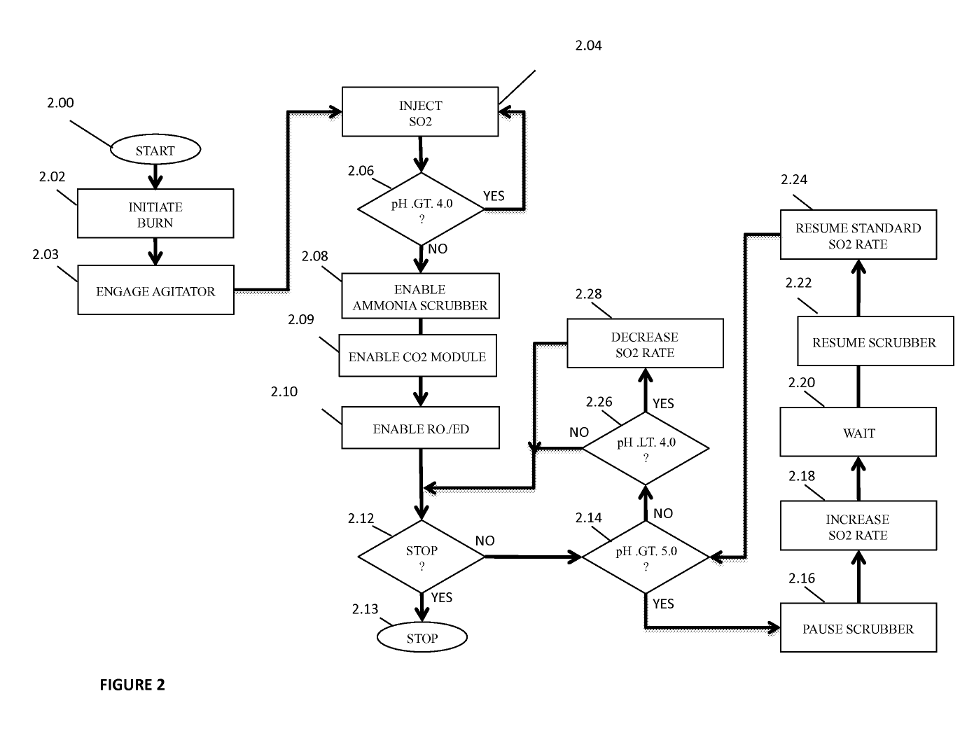

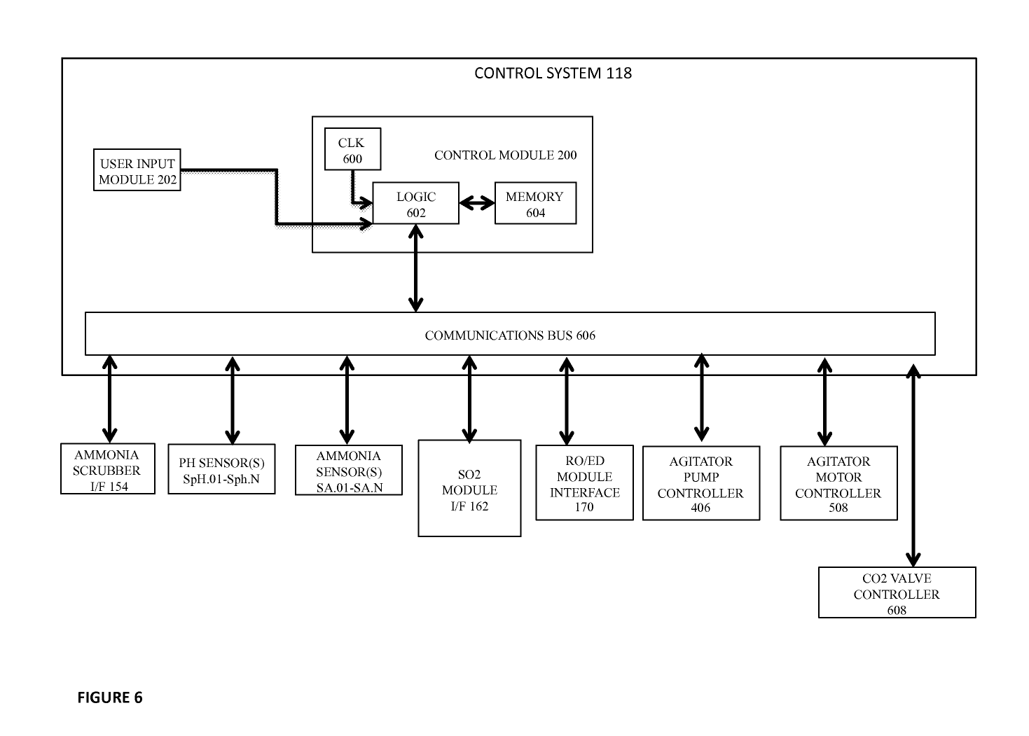

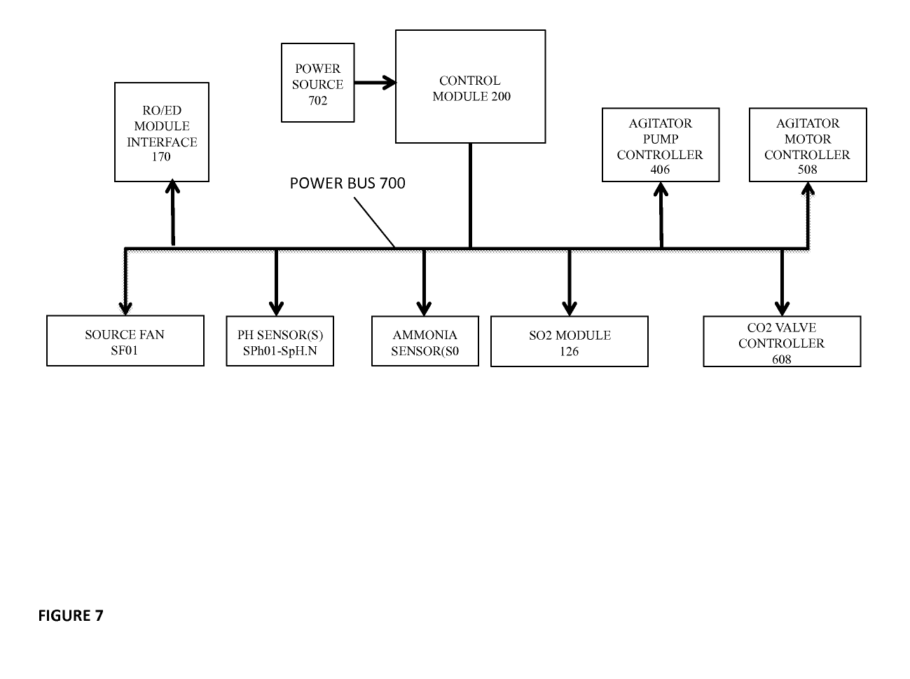

Referring now generally to the Figures and particularly to FIG. 2, FIG. 6 and FIG. 7, FIG. 2 is a software flow chart implemented by of the control system 118. In step 2.02 a command is sent from a control module 200 of the control system 118 to the sulfur dioxide module 126 to ignite the sulfur 106. In optional step 203 the control module 200 directs, and electrically powers, the agitator module 138 to agitate the source material 121 and to thereby sponsor bacterial activity that will generate gaseous ammonia 102 and optionally carbon dioxide within the enclosure 120. The volume of gaseous ammonia preferably includes molecules of NH3 and molecules of NH4+.

In step 2.02 another command is sent from the control module 200 in step 2.04 to (a.) energize motorized fluid pumps SP02 & RSP02 to circulate water volume 104 and (b.) inject the resultant sulfur dioxide gas 110 via the pressure column 158 into the water volume 104 to generate the aqueous solution 112. In step 2.06 the control module 200 accepts pH sensors SPh.01-SPh.N positioned within or proximate to the reaction chamber 124 to determine the pH of aqueous solution 112, and when the pH of the aqueous solution is not sensed to be greater than 4.0, the control system 118 directs the sulfur dioxide module 126 to simply continue inject sulfur dioxide 110 into the aqueous solution 112 until the aqueous solution 112 is measured by the pH sensors SPh.01-SPh.N to have exceeded a magnitude of approximately 4.0.

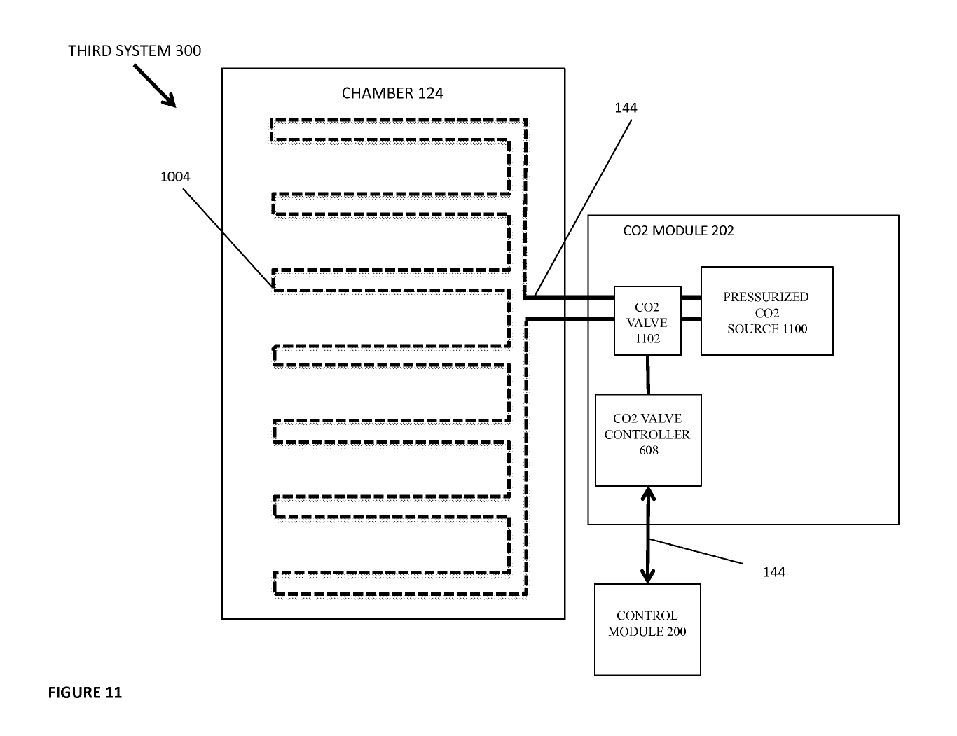

When the control module 200 receives a pH reading in step 2.06 greater than 4.0 from the pH sensors SPh.01-SPh.N, the control module 600 proceeds on to step 2.08 and energizes the ammonia scrubber 122 in step 2.08, whereby the ammonia scrubber 122 circulates the aqueous solution 112 through the ammonia scrubber 112 and exposes the aqueous solution 112 to the source ammonia gas 112. In optional step 2.09 the control system 118 directs an optional carbon dioxide module 202, as further disclosed in reference to FIG. 11, to initiate delivery of carbon dioxide into the aqueous solution 112 within the reaction chamber 124.

The control module 600 directs the RO/ED module 116 in step 2.10 to circulate the aqueous solution 112 through the RO/ED module 116 and to generate an output solution 128 for storage in the output holding tank 130.

In step 2.12 the control module 600 determines whether to continue the process of step 2.04 through 2.10, whereby portions of the aqueous solution are substantively continuously and contemporaneously circulated to and from the reaction chamber 124 and (a.) the sulfur dioxide module 126 to receive sulfur dioxide; (b.) the ammonia scrubber 122 to absorb source ammonia gas 102; and (c.) the RO/ED module 116 to filter out components, e.g., ammonium sulfate; and to generate the output solution 128. It is understood that the output solution contains (a.) a portion of the water volume 104 and (b.) one or more non-aqueous components of the aqueous solution 112 that have been separated from the aqueous solution 112 by the RO/ED module 116. The control module 200 might, for example, be programmed to proceed to step 2.13 and to shut down the first system 100 or the second system 136 when an ammonia gas detector SA01 sends a measurement that indicates that that the concentration of the source ammonia gas 102 within the atmospheric gas 114 within the enclosure 120 is less than a pre-specified amount, e.g., less than one parts per million per volume unit.

In the alternative, in step 2.12 a human operator may direct the control system via an input module 202 to cease operations and proceed to step 213 and to shut down the first system 100 or the second system 200.

The control system 118, in accordance with its structure, inputs and programming, may proceed from step 2.12 and to execute the loop of steps 2.14 through 2.28, whereby the control system 118 directs the first system 100 or the second system 136 to maintain a pH of the aqueous solution 112 approximately within a preferred range, such as approximately within the range of from 4.0 to 5.0 plus or minus five per cent.

When the control module 200 determines in step 2.14 that the pH of the aqueous solution 112 is measured to be greater than 5.0, the control system 118 proceeds on to step 2.16 and pause the activity of the ammonia scrubber 122 in circulating and exposing aqueous solution 112 for absorption of ammonia gas 102 and in step 2.18 directs the sulfur dioxide module 126 to increase the rate of introduction of sulfur dioxide 110 into the aqueous solution 110. An optional wait step 2.20 imposes a wait state of a predetermined time, and in step 2.22 the control system 118 directs the ammonia scrubber 122 to resume circulating aqueous solution 112 and causing absorption of the source ammonia gas 102 into the aqueous. The control system 118 directs the sulfur dioxide module 126 to resume a preprogrammed or pre-specified standard rate of introduction of sulfur dioxide 110 into the aqueous solution.

In the alternative, when the control module 200 determines in step 2.14 that the pH of the aqueous solution 112 is not measured to be greater than 5.0, the control system 118 proceeds on to step 2.26 and to determine if that the pH of the aqueous solution 112 is measured to be less than 4.0. When the control system 118 to determines in step 2.26 that the pH of the aqueous solution 112 is measured to be less than 4.0, the control system 118 directs the sulfur dioxide module 126 to decrease the rate of introduction of sulfur dioxide 110 into the aqueous solution 110 to a certain pre-specified or preprogrammed rate of introduction of sulfur dioxide 110 into the aqueous solution 110. The control module 200 proceeds from either step 2.26 or step 2.28 to step 2.12.

It is understood that alternative control methods to implement the invented method are made obvious to one of ordinary skill in the art in light of the present invention. In certain alternate preferred methods of the present invention, manual control, material input and/or material output may be applied, effected or enabled by a human operator to engage, disengage, turn on and/or turn-off one or more modules 116, 122 & 126, the source fan SF01, one or more motorized fluid pumps OP01, OP02, SP01-SP03 & RP01-RP04.

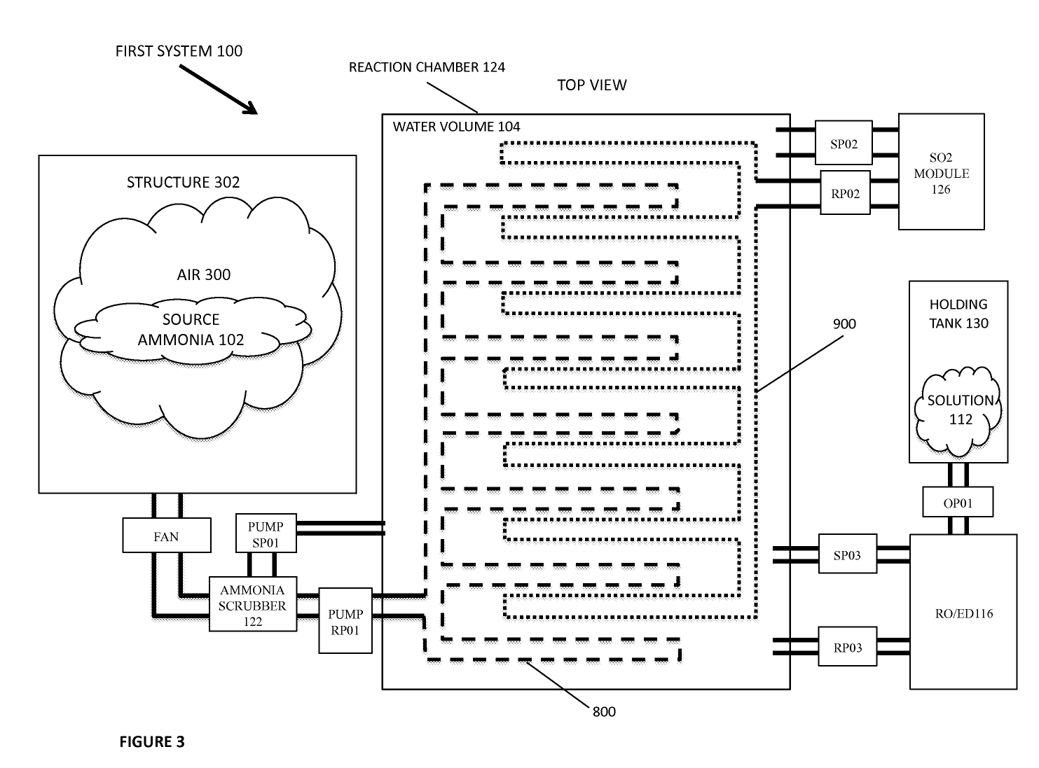

FIG. 3 is a cut-away top view of the first system 100 adapted to decontaminate the internal volume of air 300 of a substantively enclosed and contaminated structure 302, wherein the enclosed volume of air 300 includes the source gaseous ammonia 102.

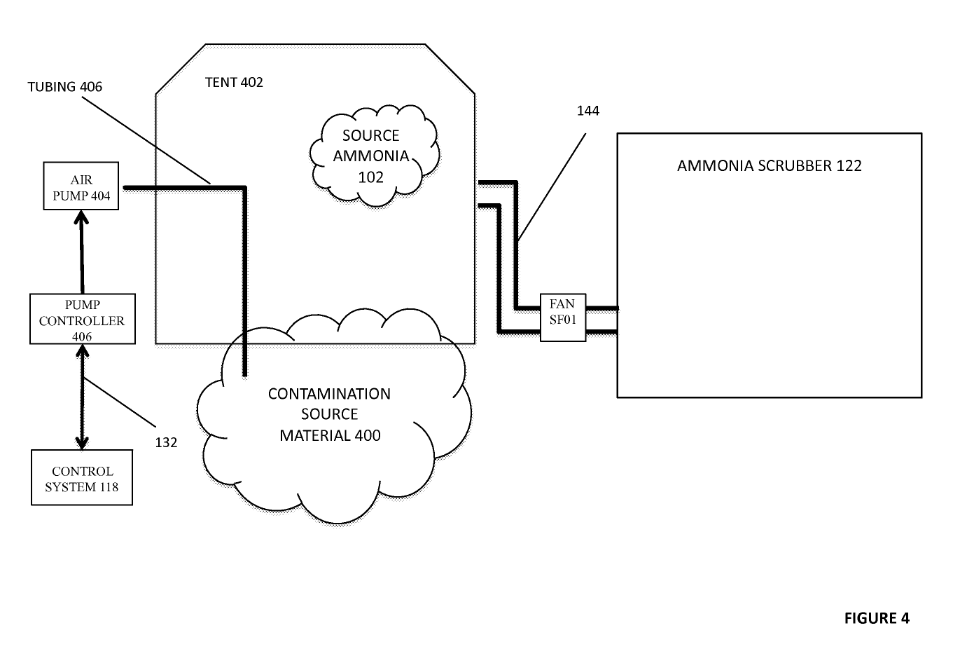

FIG. 4 is a cut-away side view of the first system 100 adapted to remediate an ammonia gas emitting and substantively liquid material 400, wherein a portable tent source enclosure 402 is placed above and around the substantively liquid material 400 and an optional motorized air pump 404 is placed and positioned to pump air into the liquid spill material 400 through a tubing 406 in order to sponsor an accelerated production of source ammonia 102 from the substantively liquid material 400 by bacterial action. An air pump controller 408 is electrically coupled with both the motorized air pump motor 404 and the power and communications bus 132, whereby the air pump controller 408 receives electrical power to energize the air pump 404 via the power and communications bus 132, wherein the control system 118 selectively and controllably delivers electrical power to the air pump controller 408.

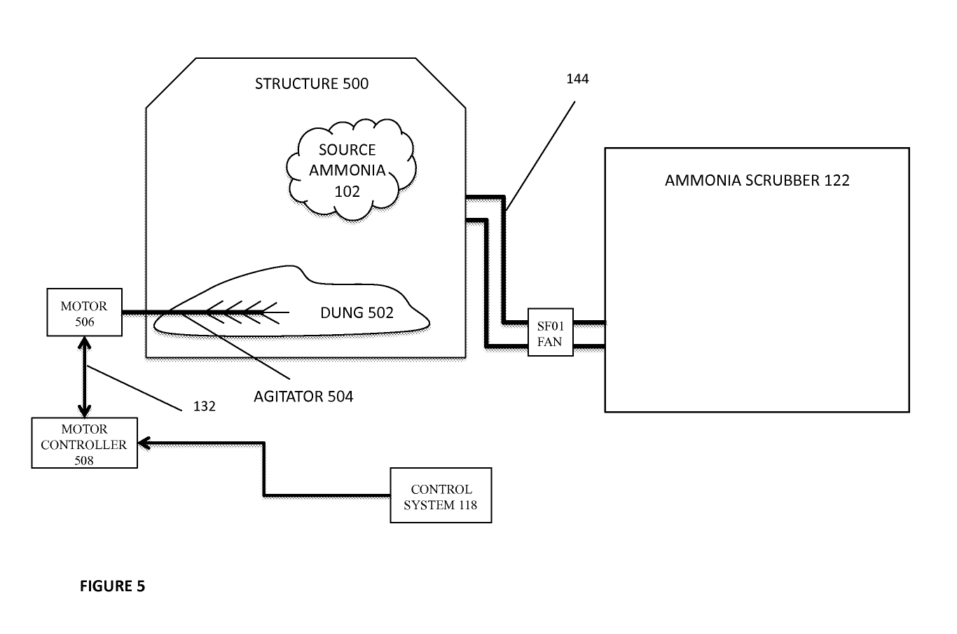

FIG. 5 is an example of the first system 100 enclosing an accumulation of a substantively solid collection of bird excrement or animal dung 500 (hereinafter, “dung 500”) housed within an enclosure 502. The dung 500 emits the source ammonia gas 102. The first system 100 is augmented with a tilling blade 504 that is rotatably coupled with an agitator motor 506. The tilling blade 504 is adapted and applied to mechanically turn over and agitate the dung 500 and thereby accelerate the production and capturing of the gaseous ammonia 102. The

An agitator motor controller 508 is electrically coupled with both the agitator motor 506 and the power and communications bus 132 and receives electrical power to energize the agitator motor 506 via the power and communications bus 132, wherein the control system 118 selectively and controllably delivers electrical power to the agitator motor 506. Additionally, alternatively or optionally, the agitator motor controller 508 may be or be comprised within an automated COMPOST-A-MATIC™ in-vessel vessel composting system as marketed by Farmer Automatic of America Inc. of Register, Ga. or other suitable motorized or automated tilling system known in the art. It is understood that the agitator module 138 may optionally or alternatively be or comprise an isolated stand-alone system that is not coupled with the power and communications bus 132 and receives an independent feed of electrical power.

FIG. 6 is a schematic diagram of an optional internal control system 118 of the first system 100 with optional modules that extend control to the second system 136. The control module 200 includes a real time clock 600 coupled with a logic controller 602. The logic controller 602 may be coupled with an optional memory 604. The logic controller 602 may be a programmable logic unit that directs the first system 100 to perform the invented method, to include the aspects of the method of FIG. 2, and/or the logic control 602 might be configured or adapted to execute programming of a software program stored within the memory 604. The control module 200 is bi-directionally communicatively coupled by means of a communication bus 606 with the ammonia scrubber interface 154, the sulfur dioxide module interface 162, the RO/ED module 116, one or more pH sensors SpH.01-Sph.N and one or more ammonia gas concentration sensors SA.01-SA.N. The communication bus 606 is preferably comprised within the power and communications bus 132.

The control module 200 may optionally or additionally be coupled with the agitator motor controller 504 and/or the agitator pump controller 406. The control module 200 may be further optionally or additionally be coupled with a carbon dioxide valve controller 608 of the carbon dioxide source module 202 of the second system 136, and as further disclosed in reference to FIGS. 6, 7 and 11.

FIG. 7 is a schematic diagram of a controlled power distribution network 700 of the control system 118 of the first system 100 of FIG. 1A and including optional elements of the second system 136. The power distribution network 700 selectively and as controlled by the control module 200 delivers electric power from an electrical power source 702 to the ammonia scrubber 122, the sulfur dioxide module 126, the RO/ED module 116, one or more pH sensors, one or more ammonia gas concentration sensors, one or more motorized fluid pumps OP01, SP01-SP-04 & RP01-RP-03, and/or the source fan SF01 of the first system 100. Additionally or alternatively, power distribution network 700 selectively and as controlled by the control module 200 delivers electric power from the electrical power source 702 to the agitator pump controller 406, the agitator motor controller 508, and/or the carbon dioxide source valve controller 608.

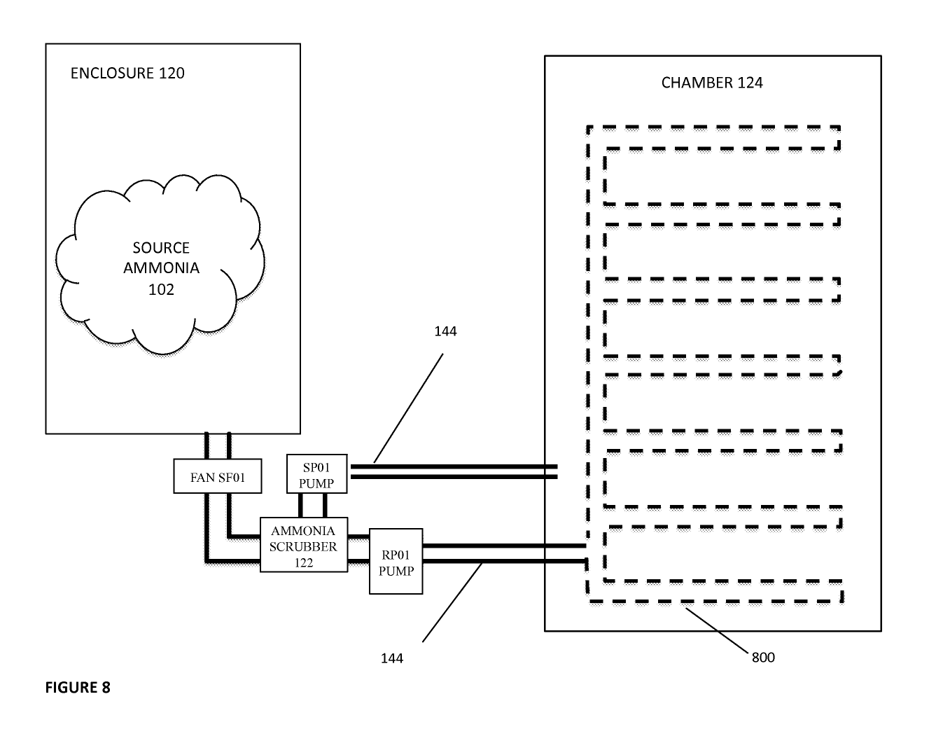

FIG. 8 is a cut-away view of an ammonia delivery perforated tubing 800 of the first system 100 that is coupled with an output port of the first return pump RP01 and returns the aqueous solution 112 from the ammonia scrubber 122 and to the reaction chamber 124. The ammonia delivery perforated tubing 800 is adapted and configured to return aqueous solution 112 from the ammonia scrubber 122 and into the reaction chamber 124, and may be or comprise perforated polyvinyl chloride piping and/or other suitable and substantively chemically inert material known in the art.

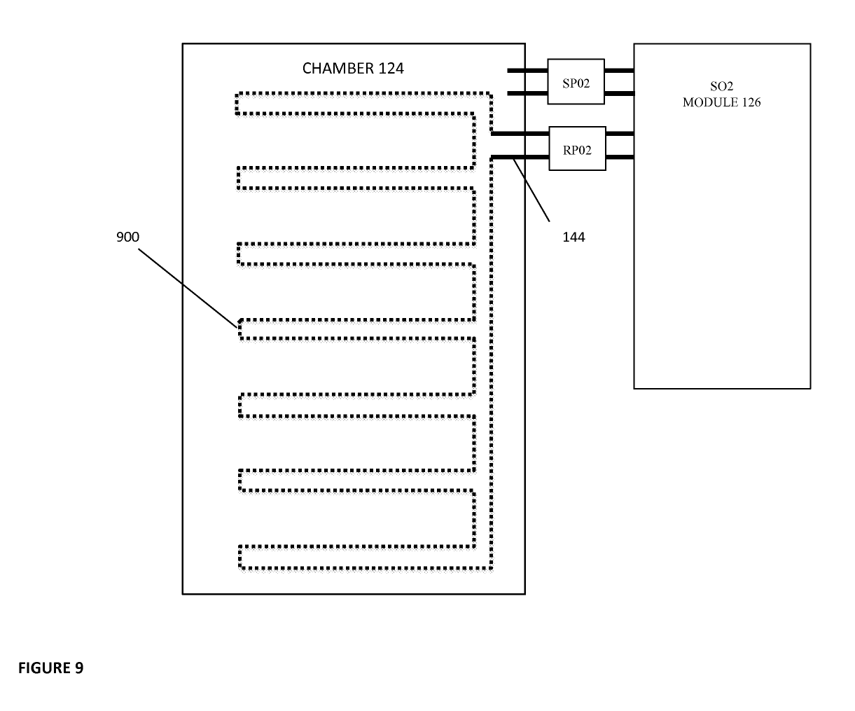

FIG. 9 is a cut-away view of a sulfur dioxide delivery perforated tubing 900 of the first system 100 that circulates and returns the aqueous solution 112 from the sulfur dioxide module 126 and to the reaction chamber 124. The sulfur dioxide delivery perforated tubing 900 is adapted and configured to return aqueous solution 112 from the sulfur dioxide module 126 and into the reaction chamber 124. The sulfur dioxide delivery perforated tubing 900 may be or comprise perforated polyvinyl chloride piping and/or other suitable and substantively chemically inert material known in the art.

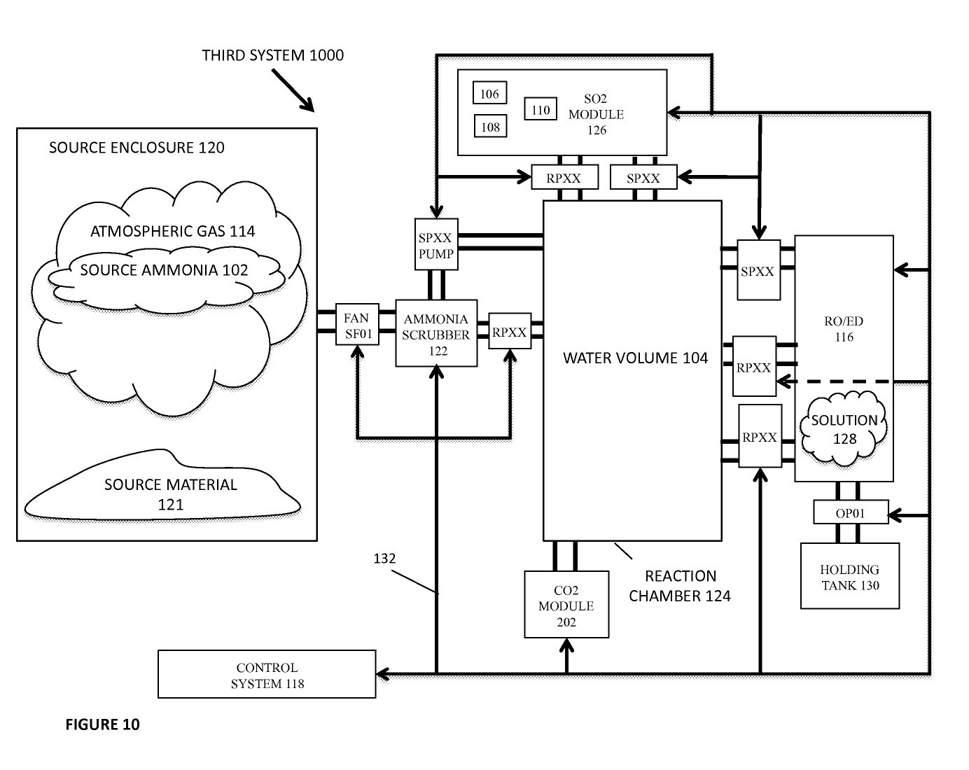

Referring now generally to the Figures and particularly to FIG. 10 and FIG. 11, FIG. 10 is a block diagram of a third alternate preferred embodiment of the present invention 1000 (hereinafter, “third system 1000”) comprising the carbon dioxide module 202 coupled with the first system 100. As disclosed in FIG. 11, the carbon dioxide module 202 comprises a source of pressurized carbon dioxide 1100 and is adapted to deliver gaseous carbon dioxide into the water volume 104 and within the reaction chamber 124 via a length of the chemically inert tubing 144. A pressure release valve 1102 is coupled with the source of pressurized carbon dioxide 1100 and a carbon dioxide delivery tubing 1004 via the length of the chemically inert tubing 144. The carbon dioxide perforated delivery tubing 1004 located within the reaction chamber 124 and is adapted to accept carbon dioxide from source of pressurized carbon dioxide 1100 and via the pressure release valve 1102. The carbon dioxide valve controller 608 controls opening and closing of the pressure release valve 1102 and receives commands and electrical power from the control module 200 via communications and power bus 132, whereby the control system 118 directs, starts, stops and controls introduction of carbon dioxide into the aqueous solution 112 from the source of pressurized carbon dioxide 1100. The carbon dioxide perforated tubing 1004 may be or comprise perforated polyvinyl chloride piping or other suitable and substantively chemically inert material known in the art.

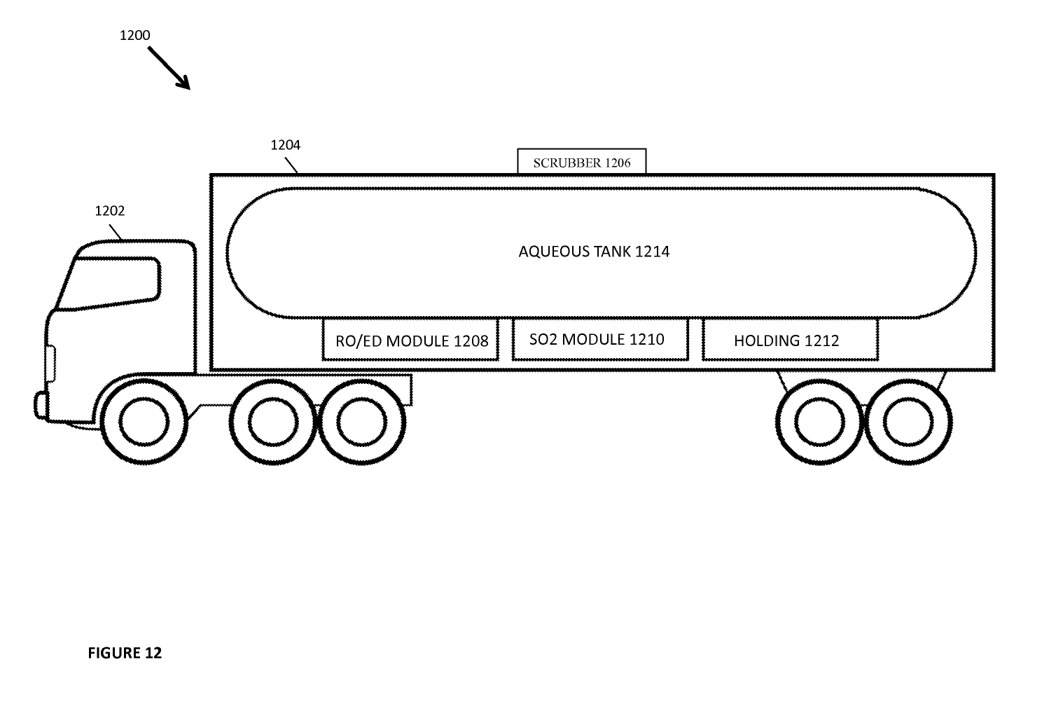

FIG. 12 is an illustration of a motorized embodiment 1200 of the first system 100. The motorized embodiment includes a motorized cab 1202 and a wheeled trailer 1204, wherein the motorized cab 1202 is adapted to detachably engage with the wheeled trailer 1204 and transport portable an ammonia gas scrubber 1206, an RO/ED module 1208, a sulfur dioxide module 1210, a components holding tank 1212 and a resultant components holding tank 1212.

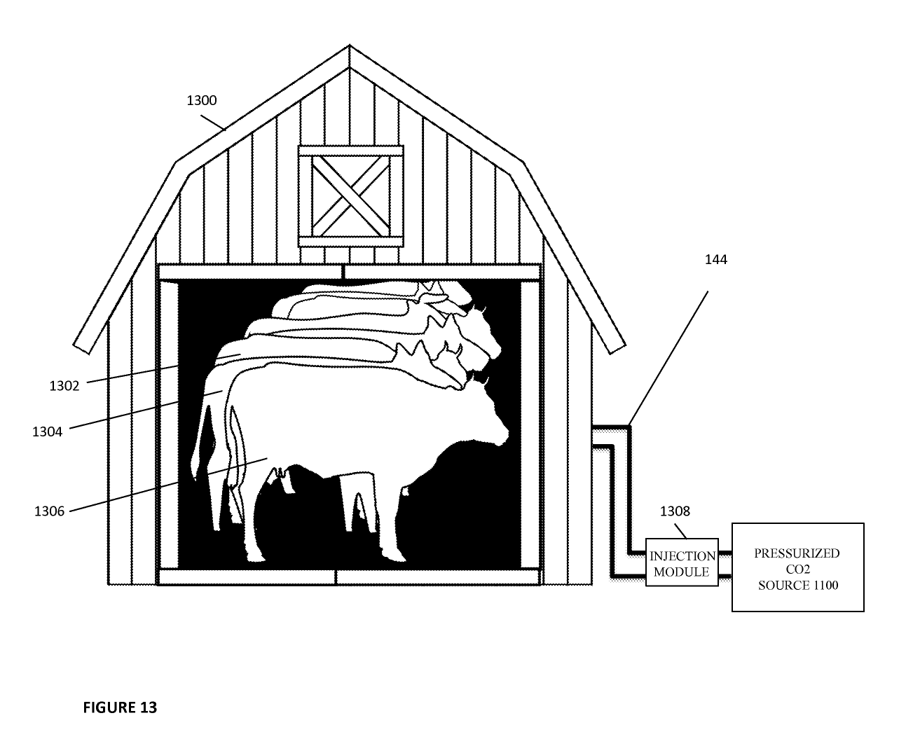

FIG. 13 is an illustration of a carbon dioxide generation 1300 module that accepts mammalian exhalation a source of gaseous carbon dioxide. An enclosed animal barn 1300 substantively encloses a plurality of mammalian livestock 1302-1306. An injection module 1308 receives carbon dioxide sourced from the mammalian livestock 1302-1306 via a length of the tubing 144. The injection module 1308 pressurizes the received carbon dioxide and injects the pressurized carbon dioxide into the pressurized carbon dioxide source 1100 via the tubing 144. The forgoing disclosures and statements are illustrative only of the Present Invention, and are not intended to limit or define the scope of the Present Invention. The above description is intended to be illustrative, and not restrictive. Although the examples given include many specificities, they are intended as illustrative of only certain possible configurations or aspects of the Present Invention. The examples given should only be interpreted as illustrations of some of the preferred configurations or aspects of the Present Invention, and the full scope of the Present Invention should be determined by the appended claims and their legal equivalents. Those skilled in the art will appreciate that various adaptations and modifications of the just-described preferred embodiments can be configured without departing from the scope and spirit of the Present Invention. Therefore, it is to be understood that the Present Invention may be practiced other than as specifically described herein. The scope of the present invention as disclosed and claimed should, therefore, be determined with reference to the knowledge of one skilled in the art and in light of the disclosures presented above.

We claim:

1. A method comprising:
a. forming a volume of fluid water;
b. burning sulfur in the presence of oxygen to form sulfur dioxide;
c. exposing the volume of fluid water to the sulfur dioxide, whereby the volume of fluid water is transformed into an aqueous solution having a pH below 5;
d. exposing the aqueous solution to an atmospheric gas volume comprising gaseous ammonia; and
e. removing a component of the aqueous solution from the aqueous solution, the component comprising nitrogen compounds.

2. The method of claim 1, wherein the component comprises ammonium sulfate.

3. The method of claim 1, wherein the component is removed from the aqueous solution in a solution comprising a portion of the aqueous solution.

4. The method of claim 1, further comprising:
f, continuing to expose the aqueous solution to the sulfur dioxide until a pH measurement of the aqueous solution of approximately four is generated; and
g. halting the exposure of the sulfur dioxide until a pH measurement of the aqueous solution of approximately five is generated; and
h. resuming exposure of the aqueous solution to the sulfur dioxide until a pH measurement of the aqueous solution of approximately four is again generated.

5. The method of claim 1, further comprising enclosing a source volume of atmosphere at approximately standard atmospheric pressure, the source volume of atmosphere additionally comprising gaseous ammonia from which the gaseous ammonia introduced into the aqueous solution is sourced.

6. The method of claim 1, wherein the volume of gaseous ammonia includes molecules of NH3 and molecules of NH4+.

7. The method of claim 1, wherein the volume gaseous ammonia is at least partially generated from mammalian dung or avian feces.

8. The method of claim 1, wherein the gaseous sulfur dioxide is pressure injected into the volume of fluid water to form the aqueous.

9. A method comprising:
a. collecting an organic waste mass containing ammonium, the waste mass comprising bacteria, wherein the bacteria generate a volume of gaseous ammonia;
b. forming a volume of fluid water;
c. burning sulfur in the presence of oxygen to form sulfur dioxide;
d. exposing the volume of fluid water to the sulfur dioxide, whereby the volume of fluid water is transformed into an aqueous solution having a pH below 5;
e. exposing the aqueous solution to the gaseous ammonia; and
f. removing a resultant component comprising a nitrogen compound from the aqueous solution.

10. The method of claim 9, wherein the resultant component comprises ammonium sulfate.

11. The method of claim 9, wherein the resultant component is at least partially removed from the aqueous solution in combination with a portion of the aqueous solution.

12. The method of claim 9, further comprising aerating the waste mass by mechanical disturbance, whereby a rate of generation of gaseous ammonia by the bacteria is increased.

13. The method of claim 12, wherein the aeration of the organic waste comprises mechanical disturbance.

14. The method of claim 9, further comprising enclosing the waste mass, whereby the gaseous ammonia is collected prior to introduction of the gaseous ammonia into the fluid water.

15. The method of claim 11, further comprising substantively removing the component from the volume fluid water while simultaneously introducing additional gaseous ammonia and additional gaseous sulfur dioxide into the volume of fluid water

16. A system comprising:
a. a volume of fluid water;
b. a sulfur burner module, the sulfur burner module adapted to form sulfur dioxide by a sustained ignition of sulfur.
c. means to inject the sulfur dioxide into the volume of fluid water, whereby the sulfur dioxide and the volume of fluid water form an acidic aqueous solution;
d. means to expose a gaseous volume comprising ammonia to the acidic aqueous solution, whereby the acidic aqueous solution absorbs ammonia; and
e. means to remove a resultant component from the aqueous solution.

17. The system of claim 16, further comprising an enclosure, the enclosure adapted to substantively contain the gaseous volume.

18. The system of claim 16, wherein the means to remove the resultant component from the aqueous solution comprises a reverse osmosis apparatus.

19. The system of claim 16, wherein the means to remove the resultant component from the aqueous solution comprises an electro-dialysis apparatus.

20. The system of claim 16, wherein resultant component comprises ammonium sulfate.
